(19) United States
(12) Patent Application Publication
(10) Pub. No.: US 2015/0064109 A1
(43) Pub. Date: Mar. 5, 2015

(54) Quantified differentiation and identification of changes in tissue by enhancing differences in blood flow and metabolic activity

(71) Applicant: Richard Max Fleming, Reno, NV (US)

(72) Inventor: Richard Max Fleming, Reno, NV (US)

(21) Appl. No.: 13/987,755

(22) Filed: Aug. 29, 2013

(51) Int. Cl. A61K 51/04

(52) U.S. Cl. CPC A61K 51/0497
USPC 424/1.65

(57) ABSTRACT

The invention provides a novel quantitative method for differentiating and identification of changes in tissue by enhancing differences in blood flow prior to administering a radiopharmaceutical, which differentially accumulates in tissue based upon differences in blood flow and metabolic activity. In one embodiment the enhancing agent is 0.852 mg per kilogram body weight dipyridamole and the radiopharmaceutical is Technetium-99m hexakis 2-methoxyisobutylisonitrile (sestamibi) and the tissue differentiation is calcification, normal, inflammatory, precancerous and cancerous breast tissue. The present invention allows differentiation between regions of calcification, nonliving or metabolically inactive tissue, normal tissue, pre-cancerous and cancerous tissue. The present invention allows for quantification of changes in tissue to determine the effect of treatment upon tissue.

### FIELD OF THE INVENTION

The present invention relates to methods for differentiating normal tissue from abnormal tissue by shifting blood flow from areas with less blood flow to areas with greater blood flow using pharmacologic vasodilitory agents equal to HDD or exercise induced effects equivalent to HDD to enhance radiopharmaceutical uptake in tissues and to “quantify the amount of uptake of these tracers dependent upon blood flow and/or tissue metabolic activity.

### BACKGROUND OF THE INVENTION

Technetium-99m hexakis 2-methoxyisobutylisonitrile (sestamibi) is an isotope that emits low levels of gamma radiation. In the body, sestamibi gets picked up by all cells, where it can be detected using physiologic imaging techniques designed to measure (“quantify”) the radiation emitted by the cells. Sestamibi is taken up by all metabolically active cells to varying degrees depending upon tissue delivery through blood flow and/or the metabolic activity of a given cell.

Sestamibi imaging (such as Miraluma™, a imaging test marketed by Bristol-Myers Squibb Medical Imaging, Inc., a subsidiary of Bristol-Myers Squibb, Inc. with headquarters at 345 Park Ave New York N.Y. 10154) is used to enhance the detection of breast cancers and is a useful adjunct to mammography. The delivery and subsequent detection of sestamibi uptake by a tumor is dependent upon (a) the delivery of the isotope to the tumor through blood flow to the tumor and (b) the metabolic activity of the cell, including but not limited to active mitochondria. It has been shown that over about 90% of sestamibi is taken up by mitochondria in an energy dependent manner. This uptake increases with the metabolic activity of the cell.

In addition to metabolic activity of the cell, the presence of sestamibi is dependent upon its delivery through the bloodstream to the region of the body being imaged. Regions of abnormality, such as inflammation, atypia and cancers, which produce angiogenic factors, have greater blood supply than do other tissues. This difference in blood supply can be augmented by the use of HDD or other pharmacologic agents or exercise effects which match HDD (defined in claims) to increase the delivery of sestamibi and other isotopes which are blood flow dependent to tissues for imaging.

Inflammatory cells typically take up sestamibi to a greater extent than normal cells, but to a lesser degree than cancer cells. This uptake demonstrates the metabolic activity of the cells to which is not entirely accounted for on the basis of mitochondrial transmembrane potential. Unfortunately, the contrast in the sestamibi uptake between normal and abnormal tissues can be is insufficient either at rest or through the use of standard dose dipyridamole (0.56 mg dipyridamole (SDD)/kilogram of patient body weight) to make accurate diagnosis regarding the presence or differentiation of abnormal tissues types. (Fleming, R M. The redistribution properties of Tc-99m isotope agents, sestamibi and myoview. Toronto Pharmacy Conference, Toronto, Canada. Sep. 27, 2012. Table 1, FIG. 3) For this reason, the use of nuclear imaging technology for detecting abnormal cells, such as cancer, has been limited. Thus a need exists for a reproducible quantifiable method to identify and differentiate between tissues enhancing differences in blood flow and metabolic activity.

Like breast cancer, the detection of coronary artery disease may also be determined by using physiologic changes in regional blood flow and nuclear imaging using Single-Photon Emission Computed Tomography (SPECT) or Positron Emission Tomography (PET) imaging or Planar imaging) methods. The ability to change coronary blood flow using high-dose dipyridamole (HDD) to detect coronary artery disease has been previously demonstrated and has been demonstrated to produce a statistically significant shift in regional blood flow to areas of the heart allowing detection of ischemic heart disease missed by SDD. Enhancement of blood flow to the heart using HDD has proven useful in unmasking heart disease through the augmentation of regional blood flow differences not possible with SDD. Specifically, HDD has been administered to patients to enhance cardiac imaging to demonstrate ischemic, infarcted and viable (metabolic function) myocardium, as well as to determine doxorubicin (marketed under the trade name Adriamycin™) induced cardiotoxicity following chemotherapy. Until now, however, the advantages of combining sestamibi or other isotopes which are dependent upon regional blood flow differences and metabolic activity imaging, with the blood flow shifting effects of HDD have not been realized in the areas of tissue differentiation of normal, inflammatory tissue and cancer diagnosis.

### SUMMARY OF THE INVENTION

The present invention provides a method for the early detection and differentiation of abnormal tissue from normal tissue. The present invention effectuates early detection of abnormal tissue using radiopharmaceutical imaging by increasing the differences in blood flow between normal and abnormal tissue, thereby enhancing the “quantification” of delivery and metabolic activity of tissue and statistically increasing the delivery of the radiopharmaceutical to all tissues, particularly abnormal tissues with greater metabolic rates. Briefly, the method of the present invention includes administering to a patient a pharmacologic agent or exercise effect that is capable of increasing the uptake of a radiopharmaceutical by shifting the delivery of the isotope to regions with greater blood flow and greater metabolic activity, which differentially accumulates in tissues with differing blood flow and/or metabolic activity and which can be statistically differentiated from each other by “quantitative measurement” of the isotope. The administration of the pharmacologic agent or equivalent physiologic exercise effect is followed by the administration of the radiopharmaceutical. Finally, the patient's tissue of interest is imaged with a radiation detector to “quantify” differences in isotope to differentiate tissue types in the patient.

In one embodiment the present invention uses a chemical agent is used to shift blood flow and delivery of the isotope to more metabolically active tissue where it will be differentially taken up by that tissue. In this embodiment HDD is a preferred agent and sestamibi is a preferred radiopharmaceutical. The physiologic effect of shifting blood flow from regions of lower blood flow to greater blood flow within tissue is associated with a “temporary increase” in the “metabolic” activity of cells. The duration of this “measurable” effect, both “quantitatively and “qualitatively” is demonstrated by the limited amount of time (HDD duration of effect is approximately 30 minutes) in which these diagnostic images may be obtained. Unlike the duration of effect demonstrated by Chiu and Crane, were the effect of “trans-membrane” mitochondrial uptake lasted 90-120 minutes, efforts to obtain diagnostic images can only be done for 30-45 minutes following HDD, while the “temporary increase” in metabolic activity and shifting of blood flow occurs; hence, these results are independent of mitochondrial activity alone which would produce images for 90-120 minutes. Therefore, imaging time is dependent upon the effect of the agent being used to differentially shift blood flow and the associated period of time were “metabolic activity” is being stimulated. After the duration of effect of HDD, both quantification and diagnostic imaging results are diminished and are non-diagnostic as demonstrated by results seen with “none statistically stimulated” flow states, such as but not limited to SDD and Miraluma™ as shown in Table 1 and FIG. 3. Fleming, R M. The redistribution properties of Tc-99m isotope agents, sestamibi and myoview. Toronto Pharmacy Conference, Toronto, Canada. Sep. 27, 2012.

### BRIEF DESCRIPTION OF THE DRAWINGS

FIG. 1 shows resting (Miraluma™) and Breast Enhanced Scintigraphy Test)(B.E.S.T.©) imaging protocols.

FIG. 2a shows examples of Miraluma™ and B.E.S.T.© images in the same patient.

FIG. 2b shows the sequence of processing images required to derive the final blue-green image and Maximal Count Activity (MCA) display of a B.E.S.T.© image.

FIG. 3 shows a comparison of MCA obtained using Miraluma™ and B.E.S.T.© imaging.

FIG. 4 shows a graphic representation of the results of MCA as seen in normal breast tissue, inflammatory tissue and breast cancer tissue, obtained using B.E.S.T.© imaging.

FIG. 5 shows differences in MCA individuals with normal, inflammatory, atypia and cancerous breast tissue.

FIG. 6 shows before and after treatment monitoring of a woman using B.E.S.T.© to “quantify” changes in breast tissue.

### DETAILED DESCRIPTION OF A PREFERRED EMBODIMENT

The present invention provides an improved method for the early detection of abnormal tissues. In one method of the present invention, the uptake of a radiopharmaceutical by abnormal tissues is increased by administering to a patient an agent which causes a shift in blood flow and isotope delivery from regions of lesser blood flow to regions with greater blood flow prior to the administration of the radiopharmaceutical isotope. This results in an increase in the delivery of the isotope to the tissue with the greatest blood flow and greater metabolic activity resulting in different levels of radiation being emitted from different tissues and, subsequently, a “quantitative” difference between tissue types, which “qualitatively” is appreciated as a clearer image of abnormal tissues as detected by physiological techniques. In one embodiment of the present invention, the uptake of the radiopharmaceutical resulting from this method yields an increased exponentially increased “quantitative” maximum count (MCA) activity in abnormal tissues compared to the uptake of the radioisotope by normal tissues.

When used to detect abnormal breast tissues, the method of the present invention will be referred to as “Breast Enhanced Scintigraphy Testing” or “B.E.S.T. ©” A description of the B.E.S.T. imaging method may be found in Fleming R M, Dooley W C, Boyd L B, Kubovy C, “Breast Enhanced Scintigraphy Testing (B.E.S.T.)—Increased accuracy in detecting breast cancer accomplished by combining breast and cardiac imaging,” 48th Annual Scientific Session of the Society of Nuclear Medicine, Toronto, Ontario, Canada, 26 Jun. 2001, the disclosure of which is incorporated herein by this reference.

The uptake of radiopharmaceuticals by cells in the body is dependent upon both the delivery of the radioactive isotope through the blood flow to that tissue and the presence of the cumulative metabolic activity of that tissue once the isotope has been delivered. Without intending to be bound to any particular theory of the invention, the inventors believe that the increase in isotope uptake in the method of this invention is due to a combination of increased blood flow in different types of tissue and the increased metabolic activity present in various types of tissue, including but not limited to leukocytes and cancerous tissue when compared to normal tissue. Cancerous tissues are extremely bloody and have increased blood flow, which can be augmented pharmacologically or physiologically only if sufficient pharmacologic or physiologic stimuli are present to shift blood flow from regions of lesser blood flow to regions of greater blood flow. SDD does not produce a satisfactory shift to accomplish this. This difference in blood flow at least partially results from angiogenic activity, which increases blood supply to the cancer providing nutrients for growth and survival. In addition, cancers and inflammatory tissue have greater metabolic activity including but not limited to mitochondrial activity when compared with than normal tissue, which has greater blood flow and metabolic activity than necrotic tissue but less than inflamed or injured tissue under these conditions.

The basic steps of the method according to the present invention include: 1) administering a pharmacologic agent or physiologic stimuli sufficient to induce a shift of blood flow from regions with lower blood flow to regions of greater blood flow to a patient; 2) administering a radiopharmaceutical to the patient which selectively accumulates in tissue based upon metabolic activity; and 3) “quantitatively” image the tissue of the patient with a radiation detector to measure the differences between tissue types in the patient. The invention may be used to increase the radiopharmaceutical uptake in a variety of abnormal tissues. Abnormal tissues are tissues other than normal healthy tissues and may include inflammatory tissue, injured tissue, infection, tissue damaged by radiation, atypia tissue demonstrating early cellular change seen in ductal carcinoma in-situ (DCIS), and cancerous tissue. When used herein, the term “atypia” means a deviation from normal or the typical. When used herein, the term “inflammatory” means a state of tissue response to injury. When used herein, the term “cancer” means a state of tissue of potentially unlimited metabolic growth that expands locally by invasion and systemically by metastasis

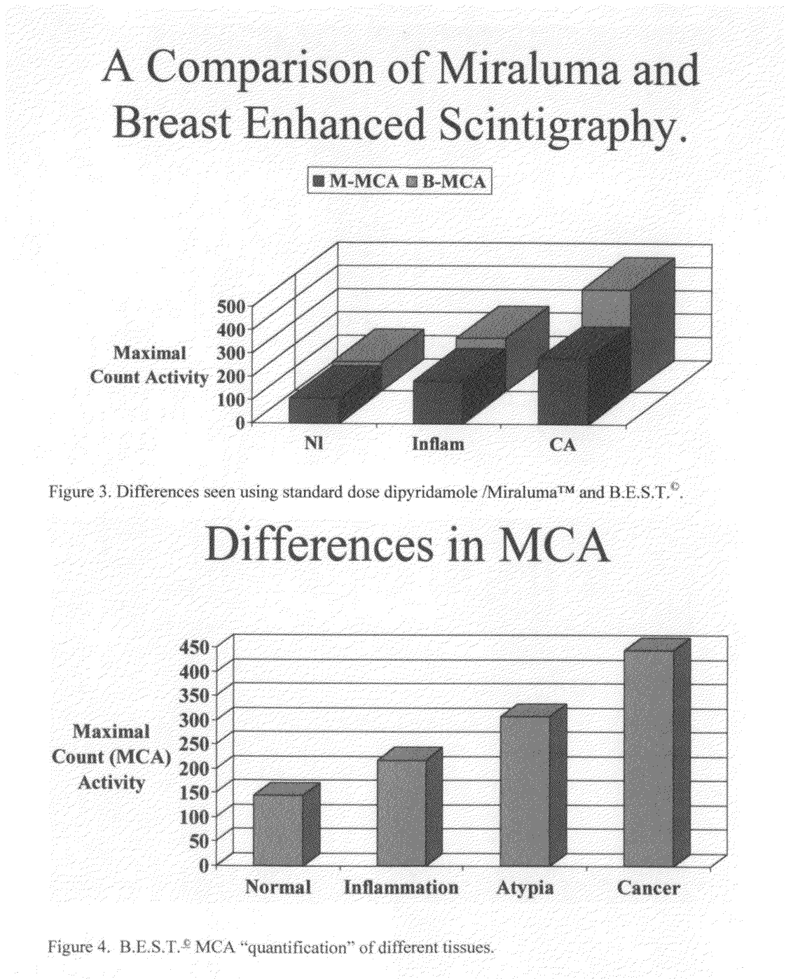

The pharmacologic agent may be any agent capable of sufficiently shifting blood flow from low flow regions to higher flow regions, thereby increasing blood flow to abnormal tissues. Dipyridamole in high dose (HDD) only is a pharmacologic agent that is particularly suited for enhancing the detection of abnormal tissues according to the present invention. However, as demonstrated in the literature (Fleming, R M. The redistribution properties of Tc-99m isotope agents, sestamibi and myoview. Toronto Pharmacy Conference, Toronto, Canada. Sep. 27, 2012. Table 1, FIG. 3) the use of standard dose (SDD) dipyridamole (0.56 mg dipyridamole per kilogram body weight of the patient) is not adequate to cause the necessary shift in blood flow. As defined and demonstrated in FIG. 3 and table 1, there is a statistically significant difference between outcomes with tissue differentiation by this method making, which demonstrated that the use of SDD was useless in tissue differentiation, while HDD was statistically significant at differentiating tissue types. In an alternative version in accordance with the principles of the present invention, physiologic exercise, adenosine, nitroglycerin and any other pharmacologic agent, which sufficiently shifts blood flow to match the effect of HDD could be utilized as the pharmacologic agents. In addition, dobutamine has shown promise in pharmacological imaging of the heart and may be a useful agent. See Fleming R M, Feldmann K M, and Fleming D M, “Comparing a High Dose Diyridamole SPECT Imaging protocol with Dobutamine and Exercise Stress Testing Protocols. Part III: Using Dobutamine to Determine Lung-to-Heart Ratios, Left Ventricular Dysfunction and a potential Viability Marker,” Intern J of Angiol 1999, 8:22-26, the disclosure of which is incorporated herein by reference.

Radiopharmaceuticals are radioactive compounds or drugs that contain one or more radioactive isotopes. Radiopharmaceuticals are taken up by cells in the body from which they emit radiation (alpha, beta or gamma rays). A biologically effective amount of a radiopharmaceutical is an amount that is sufficient to provide a detectable level of radiation when taken up by the tissue of interest in the body. Radiopharmaceuticals for use with the present invention may be any radiopharmaceutical capable of selectively accumulating in at least one type of tissue. One such radiopharmaceutical is sestamibi, which selectively accumulates in abnormal breast tissues. In an alternative version in accordance with the principles of the present invention, technetium isotopes such as but not limited to myoview, 18-flurodeoxyglucose (FDG) and fatty acid analogues could be utilized as radiopharmaceutical agents. In fact any isotope, which demonstrates the ability to differentiate tissue type based upon blood flow differences and/or metabolic function, will work.

The radiation detector may be any detection system capable of detecting the radiation emanating from the pharmaceutical within the patient's body and imaging the abnormal tissue from which the radiation originates. Such radiation detectors are well known and include, but are not limited to, single photon emission computed tomography (SPECT) detectors, positron emission tomography (PET) detectors, semiconductor detectors, geiger counters and other suitable planar imaging devices, and any other radiation detection device to be developed.

If desired, the imaging of the tissue of interest using the method of the present invention may be followed by cardiac imaging techniques utilizing the same radiopharmaceutical. Examples of such techniques are described in Fleming R M, “Chapter 29 of Atherosclerosis: Understanding the Relationship Between Coronary Artery Disease and Stenosis Flow Reserve,” Textbook of Angiology, John C. Chang Editor, Springer-Verlag New York, N.Y. 1999, pp. 381-387; Fleming R M, “Chapter 31. Nuclear Cardiology: Its Role in the Detection and Management of Coronary Artery Disease,” Textbook of Angiology, John C. Chang Editor, Springer-Verlag New York, N.Y. 1999, pp. 397-406; Fleming R M, Boyd L, Forster M, “Angina is Caused by Regional Blood Flow Differences—Proof of a Physiologic (Not Anatomic) Narrowing,” Joint Session of the European Society-American College of Cardiology, ACC 49th Annual Scientific Sessions, Mar. 12, 2000 (www.prous.com); Fleming R M, “Regional Blood Flow Differences Induced by High Dose Dipyridamole Explain Etiology of Angina,” 3rd International College of Coronary Artery Disease from Prevention to Intervention,” Lyon, France, Oct. 4, 2000; Fleming R M, Boyd L B, Kubovy C, “Myocardial perfusion imaging using high dose dipyridamole defines angina. The difference between coronary artery disease (CAD) and coronary lumen disease (CLD),” 48th Annual Scientific Session of the Society of Nuclear Medicine, Toronto, Ontario, Canada, 27 Jun. 2001; Fleming R M, “Coronary artery disease is more than just coronary lumen disease,” Am J Card 2001, 88:599-600; the disclosures of which are incorporated herein by reference.

A brief exemplary description of the B.E.S.T.© method of the present invention follows, a more detailed description of specific embodiments are illustrated in the examples below. The patient may be prepared for B.E.S.T.© imaging after having an intravenous catheter placed, through which the agents (supra) are given. The patient is then placed into a prone position for imaging with breasts hanging freely below the thorax. Once the patient is suitably comfortable, a biologically effective amount of the pharmacolgic agent is administered, intravenously. If the vasodilatory agent is HDD, a biologically effective amount will typically be not less than 0.852 mg per kg patient body weight. If the method includes exercise, the exercise portion must be performed first and be adequate to produce the physiologic effect to satisfy an exercise stress test.

After sufficient time has passed to allow the maximum shift in blood flow effect as defined by experts, package insert, the FDA, or scientifically published studies, typically about 3 to about 5 minutes, a biologically effective amount of a radiopharmaceutical is administered to the patient. The radiopharmaceutical is preferably administered intravenously by injecting it into the arm or breast contralateral to the breast to be imaged first. If other solid organ tissue is to be studied, then the biologically effective amount of isotope and time will be determined by the published literature for that specific organ. If the radiopharmaceutical is sestamibi, a biologically effective amount will typically be between about 20 and about 35 mCi.

Imaging of the breast should commence once the radiopharmaceutical has been in the patient's system long enough to allow for substantial uptake of the radiopharmaceutical by abnormal tissues in the breast tissues or the tissue being studied. Typically, for breast tissue, imaging will begin approximately 10 minutes after the onset of the study, following administration of the pharmacologic agent being used to differentially shift blood flow. The breast tissue may be imaged by any suitable scintillation detector, including a geiger counter, planar, SPECT or PET camera or other devices so approved for detecting radiation. Abnormal tissues will have greater maximal count activity (MCA) than normal tissues as “quantified” by B.E.S.T.© imaging. These abnormal tissues have the greatest blood flow and metabolic activity as measured by B.E.S.T.© MCA. The analysis of MCA may be performed using computer assessment and areas of the MCA may be displayed on a computer monitor. In a preferred embodiment of the invention, the MCA for abnormal tissues is increased without substantially altering the MCA of normal tissues, resulting in a more pronounced imaging contrast between normal and abnormal tissues, which is quantified by the MCA values.

Enhancement of the delivery of sestamibi to breast tissue according to the present invention not only allows for the distinction of “normal” tissue versus “breast cancer” but a distinction between “normal,” regions of “inflammatory” changes of the breast, regions of cellular “atypia” and breast “cancer.” There is logically a progression from “normal” breast tissue to breast “cancer” tissue. While not all regions of inflammatory changes are destined to become cancer, these regions may represent regions at greater risk of becoming a cancer, subsequently needing closer monitoring to determine if they are progressing to the development of a cancer. Clearly, the sooner a cancer is detected, the greater is the likelihood of successful treatment.

The appearance of abnormal tissue seen on B.E.S.T.© imaging also may be used to distinguish differences in the appearance of cancers and pre-cancers. Pre-cancers and cancers do not look alike when detected by physiologic methods. For example, ductal carcinoma-in-situ (DCIS) appear tubular, following the path of the milk ducts, while breast cancer appears spherical. However, once these cells have changed further, they no longer obey contact inhibition and subsequently grow into surrounding tissue producing a spherical appearance in the same way they behave in the in-vitro laboratory. These changes in appearance therefore can be used to distinguish early/pre-cancers from the next stage of infiltrating cancer. By enhancing the images using B.E.S.T.© and “quantifying” these different types of tissue which differentiates normal and different types of abnormal tissues, the present invention makes it easier to distinguish between abnormal tissue samples having various shapes and sizes.

The invention is described in greater detail in the following non-limiting examples. While the present invention has been tested and described in connection with the detection of breast cancer, the principles of the present invention are equally applicable to so-called “hard tumors” (pre-cancerous and cancerous) or other origins or locations. Thus, it is neither intended nor should the present invention be interpreted as being limited solely to the detection of breast cancer.

### Examples

In the examples that follow, two hundred and five (205) individuals who ranged in age from 27 to 88 years (51±11 years) of age were studied during a thirty three (33) month period beginning in February 1999 and ending in November 2001. The group included 201 women and 4 men. The individuals included 181 Caucasians, 5 Hispanics, 17 African American and 2 people of Mediterranean origin. No differences in outcomes were found based upon age, race or sex. Women were excluded from the study if they were taking hormone replacement therapy, were pregnant or were breast-feeding. All subjects signed consent forms prior to undergoing breast imaging.

Histopathologic information was obtained for all but 58 of the individuals studied. Tissue samples were obtained either through ductoscopy, fine needle aspiration or open biopsy and were interpreted by pathologists without knowledge of the sestamibi image results. Results were interpreted as being normal, inflammatory (including trauma, injury, infection, fibrocystic disease, radiation exposure and subsequent injury), atypia (with increasing order of cellular change progressing from hyperplasia to metaplasia/atypia to ductal carcinoma in situ) and cancer.

Two sestamibi imaging studies were conducted. The first study was conducted to demonstrate that breast cancer has increased blood flow which can be influenced by the pharmacologic blood flow shifting effect of high dose dipyridamole (HDD), and greater metabolic activity, the detection of which may be enhanced once increased delivery of sestamibi is provided through enhanced shifting of blood flow. In this study, breast tissues of ten women were studied using a conventional resting sestamibi imaging (Miraluma™) approach and SDD. These results were compared with results obtained following enhanced delivery of the isotope using HDD (B.E.S.T.© imaging). The results were also compared with biopsy data. All 10 women in the study had either an abnormality on mammography or a detectable lump on physical examination.

In a second study, one hundred and ninety five (195) (4 men, 191 women) individuals were studied using the enhanced (HDD) imaging approach. The study included four men having detectable breast lumps and 191 women, including 58 seeking additional information regarding breast disease concerns, and 133 with breast lumps and/or abnormal mammograms. The results of this study were also compared with biopsy data.

### Resting Sestamibi Breast Imaging (Miraluma™)

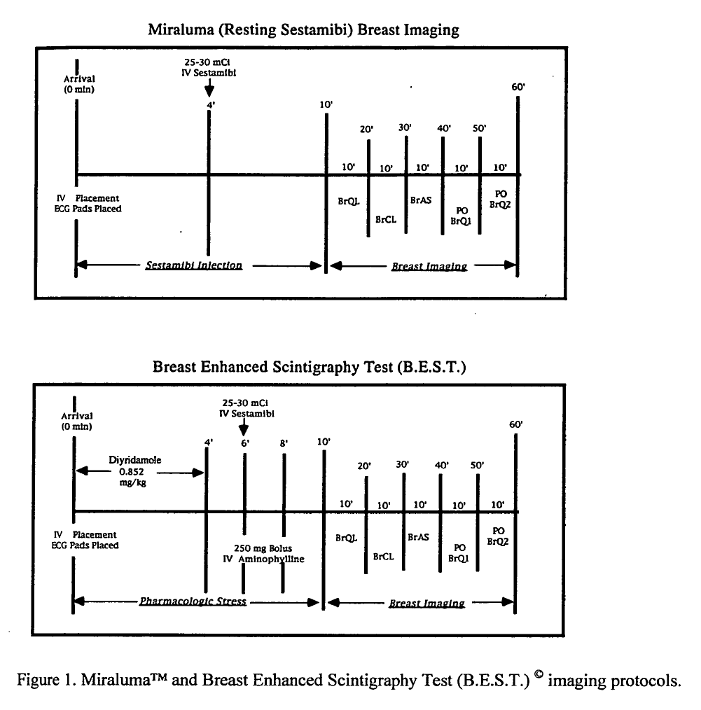

Subjects undergoing breast imaging arrived in the fasting state 15-30 minutes prior to the study. FIG. 1 shows Miraluma™ and Breast Enhanced Scintigraphy Test (B.E.S.T.©) imaging protocols. The top panel displays the protocol for resting sestamibi imaging of the breast. Subjects had an 18-20 gauge intravenous (IV) catheter placed either in the right antebrachium or the left antebrachium. The IV was placed in the contralateral arm if there is a specific question regarding a specific breast. Sestamibi was administered intravenously four minutes into the study followed by a 10-20 cc normal saline flush to assure delivery of the isotope into the venous system, with imaging of the breast beginning ten minutes into the study. A SPECT camera was positioned in a stationary (planar) position for each of the images.

Breast imaging began with the patient placed in a prone position on top of a 6-inch foam pad designed to enhance the comfort of the patient while improving breast imaging. Each side of the 6-inch pad had breast inserts held in place by hook and loop-type adhesive strips, which were removed allowing each breast to be positioned through the openings in a dependent manner without breast compression. Planar breast imaging began with the BrQL breast (lateral view of breast in question or the right breast if neither breast was specifically suspected of having an abnormality), then the BrCL breast (lateral view of the breast contralateral to BrQL). Patients were then placed on their back for the anterior (BrAS) image of both breasts. Any areas of special concern, PO BrQ1 (posterior oblique view of 1st breast noted to have abnormal activity on initial views) and PO BrQ2 (posterior oblique view of the other breast if its activity is abnormal) were then imaged.

As shown in FIG. 1, 25-30 mCi (925-1110 MBq) of Technetium-99m hexakis 2-methoxyisobutylisonitrile (sestamibi) was administered intravenously at the 4-minute mark with image acquisition beginning 6 minutes later. Ten minutes into the study, image acquisition was started, as shown in FIG. 1. All images were acquired while the patient was prone, except for the anterior (BrAS) image, which was obtained while the patient was supine.

Breast image acquisition and reconstruction was performed using a Siemens orbiter Single Photon Emission Computed Tomography (SPECT) camera with 75 photomultiplier tubes (PMTs) and a 128 by 128 matrix, available from Siemens Medical Solutions, Malvern, Pa. The images were acquired with the camera head in a stationary (planar) position. The camera, computer and software providing quantification of maximal count activity (MCA) were supplied by NC Systems of Boulder, Colo. A low-energy high-resolution (LEHR) collimator was used providing a resolution of 3.4 mm.

Cardiac imaging could have been initiated immediately after completion of the breast imaging, if desired.

### Breast Enhanced Scintigraphy Test (B.E.S.T.) Imaging

The bottom panel of FIG. 1 displays the protocol for Breast Enhanced Scintigraphy Test (B.E.S.T.©) imaging.

Subjects undergoing B.E.S.T. imaging were prepared for the study in a manner identical to that used in preparation for the Miraluma™ imaging. Subjects arrive in a fasting state 15-30 minutes prior to the study. An 18-20 gauge intravenous catheter was placed either in the right antebrachium, or in the left antebrachium. The IV was placed in the contralateral arm if a specific breast was the breast in question. As shown in FIG. 1, enhancement of blood flow shift was provided by intravenously administering not less than 0.852 milligram dipyridamole (HDD) per kilogram (mg/kg) patient body weight infused evenly over 4 minutes. The catheter was flushed with 10-20 cc of normal saline immediately after the HDD had been given to assure introduction of all of the HDD into the venous system. Two minutes later, at peak dipyridamole blood flow shifteffect, 25-30 mCi (925-1110 MBq) of is Technetium-99m hexakis 2-methoxyisobutylisonitrile (sestamibi) was administered intravenously and flushed with 10-20 cc normal saline. Image acquisition was started 10 minutes into the study as shown in FIG. 1, with the patient in a prone position. Anterior images were obtained with the patient in a supine position.

Following breast imaging, cardiac imaging was performed as described previously, providing information regarding coronary blood flow, regional wall abnormalities and left ventricular ejection fraction, all of which are useful for making further diagnostic decisions, particularly regarding the use of chemotherapy and radiation therapy. Cardiac Imaging is performed using gated images beginning immediately after completion of the breast imaging using a Siemens orbiter Single Photon Emission Computed Tomography (SPECT) camera with 75 photomultiplier tubes (PMTs) and a 128 by 128 matrix, available from Siemens Medical Solutions, Malvern, Pa. The camera, computer and software providing quantification of maximal count activity (MCA) were supplied by NC Systems of Boulder, Colo. A low-energy high-resolution (LEHR) collimator was used providing a resolution of 3.4 mm. Cardiac image acquisition required about 32 minutes using a step and shoot approach. For a description of other cardiac imaging techniques see Fleming R M, Chapter 31, “Nuclear Cardiology: Its Role in the Detection and Management of Coronary Artery Disease,” Textbook of Angiology, pp. 397-406; Fleming R M, Rose C H, Feldmann K M, “Comparing a high-dose dipyridamole SPECT imaging protocol with dobutamine and exercise stress testing protocols,” Angiology 1995, 46:547-556, which are hereby incorporated by reference.

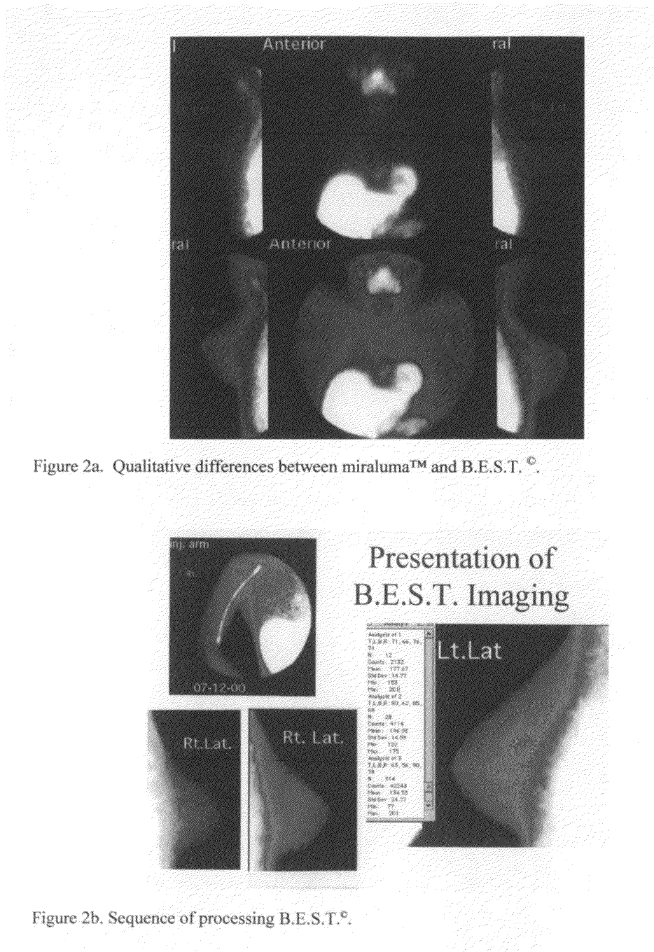

Breast imaging equipment and acquisition were identical to those used for the Miraluma™ and SDD approach. The image display for B.E.S.T.© was displayed in a blue-green format to reduce artifacts. An example of the blue-green format is shown in FIG. 2a, juxtaposed with resting images.

Following the image reconstruction and presentation of each breast image, the region of greatest maximal activity (MCA) was determined and “quantified.” MCA is a measure of detected radiation (gamma) emission acquired by the SPECT camera during the imaging process and reflects the amount of isotope present at any given time within the tissue of interest. See Fleming R M, Dooley W C, Boyd L B, Kubovy C, “Breast Enhanced Scintigraphy Testing (B.E.S.T.)—Increased accuracy in detecting breast cancer accomplished by combining breast and cardiac imaging,” 48th Annual Scientific Session of the Society of Nuclear Medicine, Toronto, Ontario, Canada, 26 Jun. 2001, the disclosure of which is incorporated herein by this reference. This assessment of MCA was performed using computer assessment of MCA as measured and displayed on the computer monitor. All readings and determination of MCA were determined for each breast prior to any knowledge of clinical, mammographic or pathologic information, which could in any way bias the results. These procedures were performed at a recognized Center of Excellence for Nuclear Procedures under the direct supervision of a physician Boarded in Nuclear Imaging.

FIG. 2b shows the sequence of processing images required to derive the final blue-green image and MCA display of a B.E.S.T.© image. Several images are displayed showing the sequence of images obtained and processed to develop the final B.E.S.T.© image with MCA measurement. The upper left image reveals the isotope (sestamibi) immediately after injection into the venous system of the right arm (inj. arm). Following image acquisition, a black and white image is displayed (Rt. Lat.) which is then converted into a blue-green image to remove visual “qualitative” artifacts present with black white images. Following this display of both breasts, the MCA is determined for each breast. In the case shown in FIG. 2b, the left breast had the greatest activity. Three regions of interest (ROIs) were measured for maximal count activity (MCA) and are displayed. Region 1 represents a smaller ROI in the upper middle breast with a MCA of 201. This ROI surrounds a milk duct as appreciated by the linear pattern of isotope distribution. The second ROI was immediately below the first and had a MCA of 175. The third ROI included the entire breast, incorporating both the first and second ROI. Consequently the MCA of the third ROI included region one which had the greatest MCA of 201.

### Comparison of the Data and Statistical Analysis

FIG. 2a shows examples of Miraluma™ and B.E.S.T.© images in the same patient. The top row of images shows (from left to right) a lateral view of the left breast (Lt. Lat), anterior (BrAS) view of both breasts, and a lateral view of the right breast (Rt. Lat). These black and white images represent the results seen with Miraluma™ imaging. The bottom row of blue-green images represents the same patient following imaging with enhanced Scintigraphy (B.E.S.T.©) imaging. The black and white Miraluma™ image was initially visually interpreted as abnormal with the appearance of increased tracer uptake (white) in the region of the left nipple; however, B.E.S.T.© revealed “normal” breast tissue using both “qualitative and quantitative” approaches.

Images were then displayed in a black and white format as shown in FIG. 2a. ROIs are then drawn around the entire breast and analysis was made for the greatest amount of tracer uptake. This greatest activity was the maximal count activity (MCA) and represents the region of breast tissue with the greatest blood flow and metabolic activity.

The outcomes of histopathologic specimens were compared with the MCA derived from sestamibi imaging. Descriptive statistical analysis of the MCAs were determined including mean±standard deviations and confidence intervals (CI) for the mean. Group comparisons were made using two-tailed t-tests to determine statistical differences defined as p-values of ≦0.05. Graphic representation of the means is shown for comparison purposes as well as raw data comparison for the histopathologic categories.

### Study 1: Results

During the first part of the study ten women underwent biopsy in addition to both Miraluma™ and B.E.S.T.© imaging. Four of the women had normal breast tissue, four had inflammatory changes and two had breast cancer. FIG. 3 shows a comparison of MCA obtained using Miraluma™ and B.E.S.T.© imaging. This bar graph represents the mean MCAs obtained for the women studied using both Miraluma™ and B.E.S.T.© imaging. The MCAs were almost identical for those with normal breast tissue, suggesting that the enhanced approach does not alter the delivery or metabolic activity of sestamibi in normal breast tissue. Individuals with inflammatory changes of the breast, however, showed statistically significant differences in MCA, which were enhanced by B.E.S.T.© imaging. These differences were even greater for individuals with breast cancer. These mean±standard deviations MCAs are shown in Table 1 and are statistically significant.

TABLE 1
MCA counts obtained using Miraluma ™, SDD and HDD.

| | Normal | Inflamatory States | Cancer |
|---|---|---|---|
| Miraluma | 107.5 ± 21.9 | 184.0 ± 19.2 | 282.5 ± 14.8 |
| SDD | 108.0 ± 20.2 | 183.5 ± 19.0 | 285.8 ± 17.0 |
| HDD/B.E.S.T. © | 125.5 ± 31.5 | 228.8 ± 24.0 | 442.0 ± 5.7 |
| p value Miraluma | NS | NS | NS |
| and SDD | | | |
| p value Miraluma | NS | p ≦ 0.05 | P ≦ 0.005 |
| and HDD/B.E.S.T. © | | | |
| p value SDD and | NS | p ≦ 0.05 | P ≦ 0.005 |
| HDD/B.E.S.T. © | | | |

The results shown in Table 1 and FIG. 3 show no statistical differences between results obtained using either the resting Miraluma™ (M) approach or the B.E.S.T.© (B) approach in normal patients. The MCA using Miraluma™ was 107.5±21.9 and was almost identical to that seen with B.E.S.T.©

In women who had inflammatory changes, Miraluma™ had a statistically lower (p<0.05) MCA compared with that seen with B.E.S.T.© imaging. The differences were more significant (p<0.005) for patients with breast cancer, where B.E.S.T.© imaging had a MCA of 442.0±5.7 while Miraluma™ had a MCA of 282.5±14.8. There were no statitistical differences between SDD and Miraluma™.

This study demonstrated that breast cancer has increased blood flow which can be affected by the blood shifting effects of HDD but not SDD, and greater metabolic activity following blood flow shift which can be further detected and quantified once increased delivery of isotope is provided through enhanced blood flow.

### Study 2: Results

In the second part of the study, the outcomes of histopathology and MCA results using B.E.S.T.© imaging were compared. FIG. 4 shows a graphic representation of the results of MCA as seen in normal breast tissue, inflammatory tissue, atypia and breast cancer tissue. MCA is plotted and displayed showing a grouping of results revealing differences between normal tissues, inflammatory tissues, atypia and breast cancer. These differences displayed an exponential increase in MCA progressing from “normal” to “atypia/cancerous” tissue.

The results shown in FIG. 4 reveal an exponential increase in MCA proceeding from “normal” to “inflammatory” to “cancer”. The MCA of patients with normal breast tissue (n=88) ranged from about 80 to 202 with an average value of 145.0±29.1. The 95% CI for “normal” breast tissue was 139 to 151. Individuals with inflammatory changes (n=77) had MCAs ranging from 130 to 298 with an average of 218.0±40.3, with a 95% CI of 209 to 227. There were 15 individuals with cellular atypia who's MCAs ranged from 209 to 333 with an average value of 307.7±29.3. The 95% CI for patients with atypia was about 292 to 323. Patients with breast cancer (n=15) had a 95% CI of 399 to 491 with an average MCA of 445.3±83.3 and a range in values from about 270 to 594. Breast cancers in this study ranged from about 4 mm to about 2 cm with the average size being about 8 to about 10 mm.

When analyzed for differences between groups, there was a statistically significant difference between normal and inflammatory tissue, inflammatory and atypia tissue, and between atypia and cancerous tissue. In each instance, the increase was statistically significant at the p<0.001 level. The raw data (FIG. 4) were analyzed following histopathologic results. The mean MCAs for each of four groups (normal, inflammatory, atypia and cancer) are displayed in table 2. These mean±standard deviation MCAs are shown in Table 2 and are statistically significant.

TABLE 2
MCA “quantitative counts obtained using B.E.S.T. © imaging.

| | | Inflamma- | Atypia (Pre- | |
|---|---|---|---|---|
| | Normal | tory States | cancerous) | Cancer |
| B.E.S.T. © | 145 ± 29.1 | 218.0 ± 40.3 | 307.7 ± 29.3 | 445.3 ± 83.3 |
| Maximal Count | | | | |
| Activity | | | | |

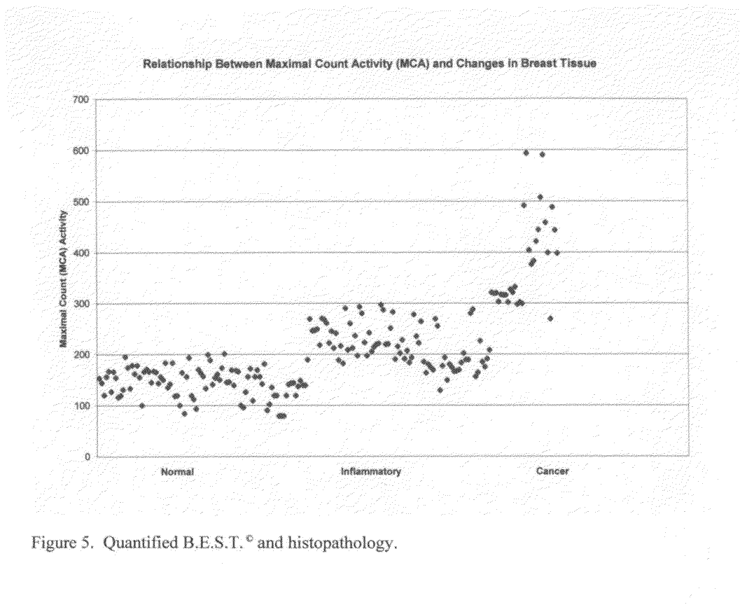

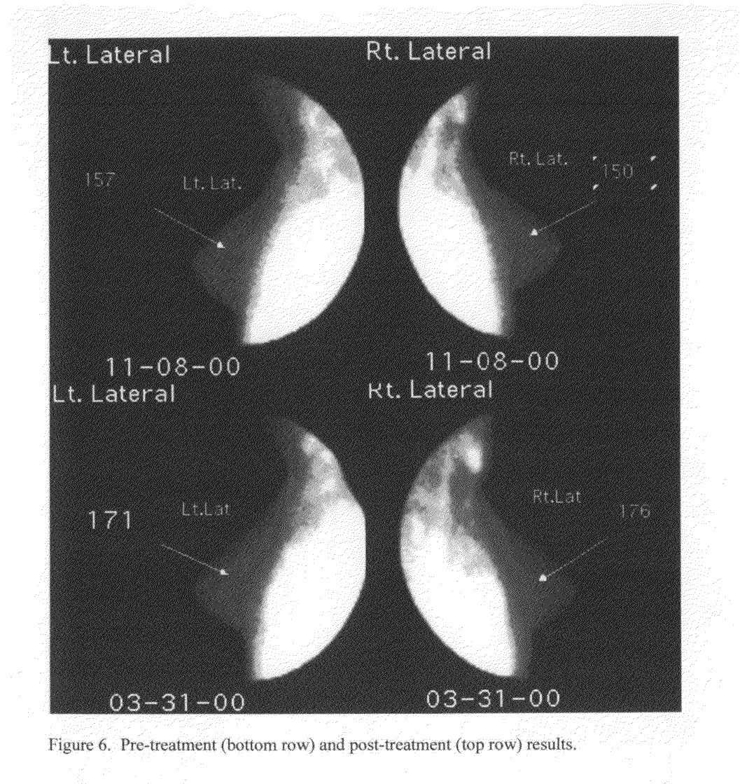

FIG. 5 shows differences in maximal count activity in individuals with normal, inflammatory, atypia and cancerous breast tissue. The results demonstrate overlap between normal and inflammatory and between inflammatory and atypia (between inflammatory and cancer) and atypia and infiltrating cancer, suggesting a probable transition as normal cells transition through a series of changes to become cancerous cells. The overall appearance of cancer differed from the visual appearance of pre-cancers (atypia, hyperplasia, etc.). Breast cancer appeared more circular consistent with a “mass effect” while atypia, hyperplasia and DCIS have values intermediate between inflammatory changes and cancer and have more of a linear pattern as shown in FIG. 2b. These findings are consistent with increased metabolic activity present in these cells and with a greater blood flow than inflammatory tissue but less than infiltrating cancers. The appearance of DCIS is typically tubular, following the ducts.

One cancer with a MCA of 270 clearly fell within the overlap MCA for upper range for inflammatory tissue and the lower range for atypia. On further inspection, the lymph nodes were negative and the tumor had little evidence of angiogenesis. It was surgically removed with no additional radiation therapy or chemotherapy recommended to the patient by her oncology team.

The findings of this study demonstrate tissue differentiation based upon shifts in blood flow obtainable only using greater than or equal to 0.852 mg dipyridamole (HDD)/kg body weight of the patient or a pharmacologic drug or exercise effect equaling that of HDD and metabolic activity temporarily enhanced by this shift in blood flow and demonstrate the ability to “quantitatively” distinguish across a continuum of breast tissue changes ranging from normal to inflammatory to atypia to cancer using sestamibi or an isotope which measures differential blood flow and/or metabolic activity of tissue, with “qualitative” appearances aiding in the differentiation of DCIS and infiltrating carcinoma. These distinctions support a transition from normal breast tissue to breast “cancer” and offer a method for distinguishing between changes in breast tissue and earlier detection and monitoring of breast cancer down to 4 mm in size. They additionally are useful for monitoring treatment response.

It should be understood that various changes and modifications to the preferred embodiment described herein would be apparent to those skilled in the art. While the present invention has been tested and is described in connection with the detection of breast cancer, the principles of the present invention are equally applicable to so-called “hard tumors” (pre-cancerous, and cancerous) of other origins or locations. For example, the present invention can be used in the detection of thymus abnormalities and coronary hearth disease where inflammation (see © TX 7-451-244) is present. Such changes and modifications can be made without departing from the spirit and scope of the present invention and without diminishing its attendant advantages. It is therefore intended that such changes and modifications be covered by the appended claims.

What is claimed is:

1. A method for quantifying, differentiating and identifying differences in tissue based upon differences in metabolic activity and blood flow, which when sufficiently enhanced physiologically and/or pharmacologically can differentially enhance blood flow in regions of inflammation, precancerous and cancerous tissue, allowing for tissue differentiation, identification and quantification:
a. The quantifying of isotope emission from tissue following this enhancement in blood flow to tissues of different metabolic activity and blood flow;
b. The quantification being performed by any detector of isotope emissions, including but not limited to planar detection, single photon emission computed tomography (SPECT) detectors, positron emission tomography (PET) detectors, semiconductor detectors, Geiger counters and/or other suitable devices;
c. From any organ system to make a diagnostic and/or treatment decision;
d. Both on an initial evaluation and subsequent evaluations;
e. Including the monitoring of treatment effectives.

2. The method for claim 1 further differentiating tissues based upon “quantitative” differences in isotope emission:
a. Which are none metabolically active (calcium, necrotic);
b. Inflammatory processes;
c. Pre-cancerous, atypia;
d. Cancerous;
e. Metastasis.

3. The method for claim 1 further differentiating tissues based upon “qualitative” differences in isotope emission:
a. Which are none metabolically active (calcium, necrotic);
b. Inflammatory processes;
c. Pre-cancerous, atypia;
d. Cancerous;
e. Metastasis.

4. The method for claim 1 further comprising significantly reducing imaging time.

5. The method for claim 1 further comprising a methodological approach which allows the incorporation of:
a. Other diagnostic studies using the same dose of isotope to measure other disease states including but not limited to inflammatory states, cellular atypia and cancerous tissue;
b. In both the diagnostic identification of these states, as well as clinical decision making and monitoring of treatment response.

6. The method for claim 1 which provides a physiologic effect not less than that produced by 0.852 mg per kilogram patient body weight of dipyridamole comprising but not limited to:
a. Treadmill, bicycle, leg ergometer, hand-held grip, adenosine, lexiscan, dobutamine, nitroglycerine, or any other physiologic/pharmacologic combination sufficient to equal the effect of 0.852 mg dipyridamole per kilogram body weight of patient.

7. The method for claim 1 which using any isotope comprising:
a. Any isotope which can produce emissions detectable by devices included in claim 1;
b. Any isotope which is dependent upon blood flow alterations to carry different quantities of isotope to different tissues comprising differential blood flow to different regions of tissue;
c. Any tissue, which can be quantified or qualitatively differentiated.

8. The method of claim 1 further comprising these claims for both humans and other animal species.
